SEQUENCE LISTING (22 sequences)

SEQ ID NO: 1 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 2 - protein, 118 aa, Artificial Sequence [sequence omitted]

SEQ ID NO: 3 - protein, 113 aa, Artificial Sequence [sequence omitted]

SEQ ID NO: 4 - protein, 8 aa, Artificial Sequence
  synthetic
GFTFNNYA

SEQ ID NO: 5 - protein, 8 aa, Artificial Sequence
  synthetic
ISGSGGTT

SEQ ID NO: 6 - protein, 11 aa, Artificial Sequence
  synthetic
AKDSNWGNFDL

SEQ ID NO: 7 - protein, 12 aa, Artificial Sequence
  synthetic
QSVLYRSNNRNF

SEQ ID NO: 8 - protein, 3 aa, Artificial Sequence
  synthetic
WAS

SEQ ID NO: 9 - protein, 9 aa, Artificial Sequence
  synthetic
QQYYTTPYT

SEQ ID NO: 10 - protein, 662 aa, Homo sapiens
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 11 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 12 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 13 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 14 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 15 - protein, 11 aa, Artificial Sequence
  synthetic
DGTRFHRQASK

SEQ ID NO: 16 - protein, 11 aa, Artificial Sequence
  synthetic
SIPWNLERITP

SEQ ID NO: 17 - protein, 8 aa, Artificial Sequence
  synthetic
SIPWNLER

SEQ ID NO: 18 - protein, 11 aa, Artificial Sequence
  synthetic
SIPWNLERIIP

SEQ ID NO: 19 - protein, 8 aa, Artificial Sequence
  synthetic
AKDPWRLP

SEQ ID NO: 20 - protein, 9 aa, Artificial Sequence
  synthetic
AARRGYLTK

SEQ ID NO: 21 - protein, 8 aa, Artificial Sequence
  synthetic
SKEAWRLP

SEQ ID NO: 22 - protein, 9 aa, Artificial Sequence
  synthetic
AARRGYVIK

(19) United States
(12) Patent Application Publication
(10) Pub. No.: US 2015/0335615 A1
(43) Pub. Date: Nov. 26, 2015

(54) CHOLESTEROL-LOWERING COMPOUNDS IN COMBINATION WITH LIPID METABOLISM-ALTERING COMPOUNDS OF NON-ABSORBABLE SUGARS, COMPOUNDS THAT CONVERT NH3 TO NH4+, OR HYDROGEN-GENERATING COMPOUNDS FOR THE TREATMENT OF HIGH CHOLESTEROL AND INFLAMMATION

(71) Applicants: Toshihisa Kawai, Brookline, MA (US); Kenneth I. Kohn, West Bloomfield, MI (US)

(72) Inventors: Toshihisa Kawai, Brookline, MA (US); Kenneth I. Kohn, West Bloomfield, MI (US)

(21) Appl. No.: 14/758,903

(22) PCT Filed: Jan. 6, 2014
(86) PCT No.: PCT/US14/10329

§ 371 Date: Jul. 1, 2015

Related U.S. Application Data
(60) Provisional application No. 61/748,944, filed on Jan. 4, 2013.
(60) Provisional application No. 61/750,862, filed on Jan. 10, 2013.
(60) Provisional application No. 61/761,959, filed on Feb. 7, 2013.
(60) Provisional application No. 61/836,893, filed on Jun. 19, 2013.

(51) Int. Cl. A61K 31/40; A61K 39/395; A61K 33/00; A61K 31/7016

(52) U.S. Cl. CPC A61K 31/40; A61K 31/7016; A61K 39/3955; A61K 33/00

(57) ABSTRACT

A composition for treating high cholesterol, including synergistically effective amounts of a cholesterol-lowering agent and a lipid metabolism-altering compound chosen from a non-absorbable sugar, a compound that converts NH3 to NH4+, a hydrogen-generating compound, and combinations thereof. A composition for treating high cholesterol, including synergistically effective amounts of a statin chosen from atorvastatin, cerivastatin, fluvastain, lovastatin, mevastatin, pitavastatin, pravastatin, rosuvastatin, and simvastatin, and lactulose. A method of lowering cholesterol by administering a synergistically effective amount of the composition to an individual, and lowering cholesterol in the individual.

### BACKGROUND OF THE INVENTION

1. Technical Field

The present invention relates to compositions and methods for treating high cholesterol and inflammation. More specifically, the present invention relates to cholesterol-lowering agents in combination with lipid metabolism-altering compounds for treating high cholesterol and inflammation.

2. Background Art

High cholesterol results from the build-up of fatty deposits in blood vessels, tightening the space available for blood to flow through arteries. This increases the risk of heart attack as well as stroke. High cholesterol can be a genetic condition but also is often the result of an unhealthy diet and little exercise.

There are many cholesterol-lowering medications and lifestyle changes available to those with high cholesterol. Individuals can switch to a healthy diet and start exercising. Statins (HMG-CoA reductase inhibitors) are also commonly prescribed in order to lower cholesterol, as are bile-acid-binding resins, cholesterol absorption inhibitors, and combinations thereof. Prescribed statins include atorvastatin, fluvastatin, lovastatin, pitavastatin, and simvastatin. Common side effects include muscle pain, stomach pain, constipation, nausea, and diarrhea, as well as adverse effects on the user's liver. Some of the more serious side effects of statins are myositis, elevated levels of CPK, and rhabdomyolysis. Each of these conditions involves inflammation of the muscles, from mild to extreme. Therefore, many individuals have to discontinue use of statins to reduce their cholesterol if such conditions are present.

There are several conditions that result in elevated levels of ammonia in the body, i.e. hyperammonemia). One condition, hepatic encephalopathy, is caused by liver damage or disease and results in an elevated level of ammonia in the body due to the inability of the liver to remove this toxic substance. Ammonia is produced by the body and intestinal bacteria when proteins are digested. Lactulose, a non-absorbable disaccharide, is generally prescribed for this condition to prevent intestinal bacterial from creating ammonia. Neomycin, rifaximin, and branched-chain amino acids are also prescribed for hepatic encephalopathy. Other treatments of hyperammonemia include sodium benzoate and phenylacetate.

Lactulose has been studied in several situations. Luo, et al. (Eur J Gastroenterol Hepatol) showed that lactulose had significant benefits for patients suffering from minimal hepatic encephalopathy (MHE) as compared to placebo or no intervention. Lactulose reduced the risk of no improvement in neuropsychological tests, prevented the progression to overt hepatic encephalopathy, reduced blood ammonia levels, and improved health-related quality of life.

Jia, et al. (World J Gastroenterol. 2005 Feb. 14; 11(6):908-11) showed that probiotic treatment is comparable to lactulose in treating minimal hepatic encephalopathy. Either probiotics or lactulose significantly lowered the level of hyperammonemia and hyper-endotoxemia, lightened centrolobular necrotic areas as well as inflammatory reaction in the liver of rats, normalized the latency of BAEP, and decreased the incidence of MHE.

Weber F L Jr. (Scand J Gastroenterol Suppl. 1997; 222:83-7) shows that metabolism by the enteric flora is necessary for the mechanism of action of lactulose. When the intestinal flora metabolizes lactulose, bacterial incorporation of nitrogen increases, as does the bacterial mass. The presence of a carbohydrate and the acidic environment caused by the production of organic acids also act to reduce the breakdown of other nitrogen-containing compounds to ammonia and other potential cerebral toxins. Other non-absorbable saccharides, particularly those contained in dietary fiber, appear to have effects similar to those of lactulose.

Therefore, there remains a need for a cholesterol-lowering agent that improves cholesterol levels while reducing the risk of adverse reactions, and allowing populations that currently do not respond to cholesterol-lowering agents to receive treatment. There also remains a need for an agent that is able to reduce cholesterol on its own, as well as work synergistically with other cholesterol-lowering agents to reduce cholesterol while also reducing the side effect of inflammation associated with these agents. There remains a need for an agent that can convert excess ammonia in the body to a useful product. There also remains a need for a composition that can suppress weight gain.

### SUMMARY OF THE INVENTION

The present invention provides for a composition for treating high cholesterol, including synergistically effective amounts of a cholesterol-lowering agent and a lipid metabolism-altering compound chosen from a non-absorbable sugar, a compound that converts NH3 to NH4+, a hydrogen-generating compound, and combinations thereof.

The present invention also provides for a composition for treating high cholesterol, including synergistically effective amounts of a statin chosen from atorvastatin, cerivastatin, fluvastain, lovastatin, mevastatin, pitavastatin, pravastatin, rosuvastatin, and simvastatin, and lactulose.

The present invention provides for a method of lowering cholesterol, in particular ox-LDL, by administering a synergistically effective amount of a composition including a cholesterol-lowering agent and a lipid metabolism-altering compound chosen from a non-absorbable sugar, a compound that converts NH3 to NH4+, a hydrogen-generating compound, and combinations thereof to an individual, and lowering cholesterol in the individual.

### DESCRIPTION OF THE DRAWINGS

Other advantages of the present invention are readily appreciated as the same becomes better understood by reference to the following detailed description when considered in connection with the accompanying drawings wherein:

FIG. 1 is a diagram showing mice fed with a high fat diet having elevated levels of oxidative LDL;

FIG. 2 is a diagram of a hyperlipidemia/atherosclerosis model;

FIG. 3 is a diagram of pathways of atherosclerosis;

FIG. 4 is a graph of the effect of hydrogen on hydrogen sulfide;

FIGS. 5A-5F are photographs of atherosclerosis region in aorta of wild type and LDLR-KO;

FIG. 6 is a chart of the level of plaque deposition in experiment groups;

FIG. 7 is a chart showing fatty liver development in experiment groups;

FIG. 8A is a graph of HDL (good cholesterol) levels, and FIG. 8B is a graph of LDL (bad cholesterol) levels in experiment groups;

FIG. 9 is a chart of the ratio of weight increase with mice on different diets and shows that lactulose has an adiposity protective effect;

FIGS. 10A and 10B are graphs showing the amount of Ox-LDL in serum for various groups of mice;

FIGS. 11A-11D are photographs of atherosclerosis plaque evaluation;

FIG. 12 is a graph of the amount of lipid positive areas in mice;

FIGS. 13A-13F are photographs of atherosclerotic plaque in the aorta; and

FIG. 14 is the chemical structure of atorvastatin calcium.

### DETAILED DESCRIPTION OF THE INVENTION

The present invention is generally directed to compositions and methods that are useful for treating high cholesterol and/or inflammation. The compositions are also useful in suppressing weight gain. The compositions are particularly suited to alter lipid metabolism, lower Ox-LDL, prevent atherosclerosis, prevent fatty liver development, and prevent metabolic syndrome effects due to an individual's high fat diet.

“Hydrogen” as used herein, refers to the composition H₂ (also written herein as “H2”), but can also include molecular hydrogen (H) and any composition capable of releasing hydrogen. In other words, H2 molecules per se can be administered, a prodrug able to release H2, or a compound that can cause the release of H2 within the body can be administered, as further described below.

“Lowering cholesterol” as used herein, refers to the ability of the composition to lower elevated cholesterol levels in an individual to a healthy level of cholesterol, or an otherwise reduced level of cholesterol.

“Suppressing weight gain” as used herein, refers to the ability of the composition to prevent individuals from gaining excessing weight, or additional weight. In other words, the composition aids at maintaining an individual at a healthy weight.

More specifically, the present invention is directed to a composition of a cholesterol-lowering agent in combination with a lipid metabolism-altering compound that generates increased hydrogen in the body, converts NH3 to NH4+, and/or is a non-absorbable sugar, each in synergistically effective amounts.

Most preferably, the cholesterol-lowering agent is a statin. Statins (HMG-CoA reductase inhibitors) are one type of drug that produce an unwanted side effect of inflammation (such as myositis, myalgia, and rhabdomyolysis). Statins are generally indicated for lowering cholesterol levels in blood and for preventing heart attacks and stroke. Different statins include atorvastatin, cerivastatin, fluvastain, lovastatin, mevastatin, pitavastatin, pravastatin, rosuvastatin, simvastatin, and statin combinations with other agents. Any of these statins can be used in the composition of the present invention.

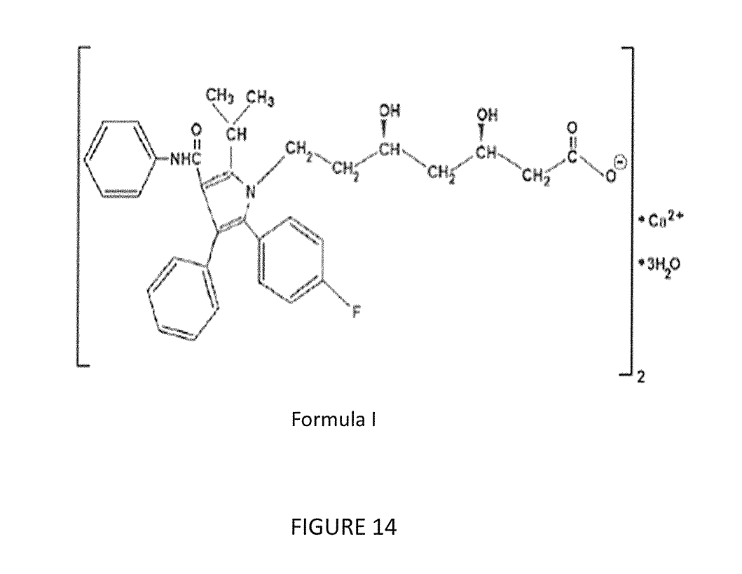

One particular statin that can be used with the lipid metabolism-altering compound is atorvastatin (LIPITOR®, Pfizer). Atorvastatin is also known as (3R,5R)-7-[2-(4-fluorophenyl)-3-phenyl-4-(phenylcarbamoyl)-5-propan-2-ylpyrrol-1-yl]-3,5-dihydroxyheptanoate, and is described in U.S. Pat. Nos. 4,681,893; 5,273,995; 5,686,104; 6,126,971; and 5,969,156. Atorvastatin calcium is shown in FIG. 14 in Formula I.

Atorvastatin can be administered in oral form in doses of 10 to 80 mg/day; however, due to synergism with the lipid metabolism-altering compound, a lower dose can be preferred and normal side effects experienced can be reduced or eliminated. For example, inflammation normally experienced with administration of atorvastatin can be reduced or eliminated, allowing many individuals who previously had side effects from atorvastatin to use atorvastatin in combination with the lipid metabolism-altering compound safely. Any other administration methods as described herein can also be used. Dosing for any of the other statins described above can be similar to that for atorvastatin.

Alternatively, any other suitable cholesterol-lowering agent can also be used. For example, the cholesterol-lowering agent can be any lipid-lowering antibody, i.e. any antibody that has the ability to effect cholesterol metabolism to reduce high cholesterol in an individual. The lipid-lowering antibody can also be used in combination with other cholesterol-lowering agents in the composition, such as the statins listed above.

Most preferably, the lipid-lowering antibody is the monoclonal antibody (MAb) anti-PCSK9. The anti-PCSK9 MAb can be any suitable antibody that binds with PCSK9, or a portion(s) thereof, in order to block its mechanism of action, but can also preferably be any specific anti-PCSK9 described below. Preferably, the anti-PCSK9 MAb is injected intravenously, but can be administered in any method described herein.

Proprotein convertase subtilisin/kexin type 9 (PCSK9) plays a critical role in cholesterol metabolism by controlling the levels of LDL particles that circulate in the bloodstream. PCSK9 increases plasma LDL cholesterol by promoting degradation of the LDL receptor, which mediates LDL endocytosis in the liver, the major route of LDL clearance from circulation. Anti-PCSK9 MAb blocks PCSK9 so that LDL levels are reduced. The ammonium ion (NH4+) can protect PCSK9-mediated LDL-receptor (LDLR) degradation. The lipid metabolism-altering compound, especially lactulose described below, is able to convert toxic ammonia (NH3) derived from gut bacteria to ammonium ion (NH4+), both in the gut as well as systemically throughout the body. Elevated ammonium ion maintains the good LDLR that captures and destroys bad LDL in the liver. Therefore, the combination of the lipid metabolism-altering compound and anti-PCSK9 MAb is synergistic and provides additive effects in lowering high cholesterol. Further synergy can exist when the lipid metabolism-altering compound, lipid-lowering antibody (anti-PCSK9 MAb), and statins are used in combination.

U.S. Pat. No. 8,062,640 to Sleeman, et al. discloses a human antibody or antigen-binding fragment of a human antibody that specifically binds and inhibits human proprotein convertase subtilisin/kexin type 9 (hPCSK9) characterized by the ability to reduce serum LDL cholesterol by 40-80% over a 24, 60 or 90 day period relative to predose levels, with little or no reduction in serum HDL cholesterol and/or with little or no measurable effect on liver function, as determined by ALT and AST measurements.

The anti-hPCSK9 antibody or antigen-binding fragment of an antibody can bind an epitope within the catalytic domain of hPCSK9, which is about 153 to 425 of SEQ ID NO: 755 (SEQ ID NO: 1 herein); more specifically, an epitope from about 153 to about 250 or from about 250 to about 425; more specifically, the antibody or antibody fragment of the invention binds an epitope within the fragment from about 153 to about 208, from about 200 to about 260, from about 250 to about 300, from about 275 to about 325, from about 300 to about 360, from about 350 to about 400, and/or from about 375 to about 425.

The anti-hPCSK9 antibody or antigen-binding fragment of an antibody can bind an epitope within the propeptide domain (residues 31 to 152 of SEQ ID NO: 755 (SEQ ID NO: 1 herein)); more specifically, an epitope from about residue 31 to about residue 90 or from about residue 90 to about residue 152; more specifically, the antibody or antibody fragment binds an epitope within the fragment from about residue 31 to about residue 60, from about residue 60 to about residue 90, from about residue 85 to about residue 110, from about residue 100 to about residue 130, from about residue 125 to about residue 150, from about residue 135 to about residue 152, and/or from about residue 140 to about residue 152.

The anti-hPCSK9 antibody or antigen-binding fragment of an antibody can bind an epitope within the C-terminal domain, (residues 426 to 692 of SEQ ID NO: 755 (SEQ ID NO: 1 herein)); more specifically, an epitope from about residue 426 to about residue 570 or from about residue 570 to about residue 692; more specifically, the antibody or antibody fragment binds an epitope within the fragment from about residue 450 to about residue 500, from about residue 500 to about residue 550, from about residue 550 to about residue 600, and/or from about residue 600 to about residue 692.

The antibody or antibody fragment can bind an epitope that includes more than one of the enumerated epitopes within the catalytic, propeptide or C-terminal domain, and/or within two or three different domains (for example, epitopes within the catalytic and C-terminal domains, or within the propeptide and catalytic domains, or within the propeptide, catalytic and C-terminal domains.

The antibody or antigen-binding fragment can bind an epitope on hPCSK9 comprising amino acid residue 238 of hPCSK9 (SEQ ID NO: 755 (SEQ ID NO: 1 herein)).

The mAbs can be full-length (e.g., an IgG1 or IgG4 antibody) or may comprise only an antigen-binding portion (e.g., a Fab, F(ab′).sub.2 or scFv fragment), and may be modified to affect functionality, e.g., to eliminate residual effector functions (Reddy et al. (2000) J. Immunol. 164:1925-1933).

The anti-PCSK9 MAb can include heavy and light chain CDR sequences from the HCVR (heavy chain variable region) and LCVR (light chain variable region) sequence pair having SEQ ID NOs: 90/92 (SEQ ID NOs: 2 and 3 herein). The anti-PCSK9 MAb can include heavy and light chain CDR sequences having SEQ. ID NOs; 76, 78, 80, 84, 86, and 88 (SEQ ID NOs: 4, 5, 6, 7, 8, and 9 herein). The anti-PCSK9 MAb can include an HCVR having the amino acid sequence, of SEQ ID NO:90 (SEQ ID NO: 2 herein) and an LCVR having the amino acid sequence of SEQ ID NO: 92 (SEQ ID NO: 3 herein).

U.S. Pat. Nos. 8,030,457, 8,168,762, U.S. Patent Application Publication Nos. 2011/0027287, 2012/0020975, 2012/0027765, 2012/0213797, and 2012/0251544 to Jackson, et al. disclose antigen binding proteins that interact with PCSK9 to decrease the LDLR lowering effect of PCSK9 on LDLR, the PCSK9 preferably having the amino acid sequence of SEQ ID NO: 1 (SEQ ID NO: 10 herein), or variants or mutations thereof.

An isolated neutralizing antigen binding protein or human monoclonal antibody that binds to a PCSK9 protein including the amino acid sequence of SEQ ID NO: 1 (SEQ ID NO: 10 herein) as described in U.S. Pat. No. 8,030,457, wherein the neutralizing antigen binding protein includes: a heavy chain polypeptide including the following complementarity determining regions (CDRs): a heavy chain CDR1 that is a CDR1 in SEQ ID NO: 49 (SEQ ID NO: 11 herein); a heavy chain CDR2 that is a CDR2 in SEQ ID NO: 49 (SEQ ID NO: 11 herein); a heavy chain CDR3 that is a CDR3 in SEQ ID NO: 49 (SEQ ID NO: 11 herein) and a light chain polypeptide including the following CDRs: a light chain CDR1 that is a CDR1 in SEQ ID NO: 23 (SEQ ID NO: 12 herein); a light chain CDR2 that a CDR2 in SEQ ID NO: 23 (SEQ ID NO: 12 herein); and a light chain CDR3 that is a CDR3 in SEQ ID NO: 23 (SEQ ID NO: 12 herein).

An isolated neutralizing antigen binding protein or human monoclonal antibody that binds to a PCSK9 protein of the amino acid sequence of SEQ ID NO: 1 (SEQ ID NO: 10 herein) can be used as described in U.S. Pat. No. 8,168,762, wherein the neutralizing antigen binding protein includes a heavy chain polypeptide including the following complementarity determining regions (CDRs): a heavy chain CDR1 that is a CDR1 in SEQ ID NO: 67 (SEQ ID NO: 13 herein); a heavy chain CDR2 that is a CDR2 in SEQ ID NO: 67 (SEQ ID NO: 13 herein); a heavy chain CDR3 that is a CDR3 in SEQ ID NO: 67 (SEQ ID NO: 13 herein); and a light chain polypeptide including the following CDRs: a light chain CDR1 that is a CDR1 in SEQ ID NO: 12 (SEQ ID NO: 14 herein); a light chain CDR2 that a CDR2 in SEQ ID NO: 12 (SEQ ID NO: 14 herein); and a light chain CDR3 that is a CDR3 in SEQ ID NO: 12 (SEQ ID NO: 14 herein).

WO2011/027257 to Champion, et al. discloses an immunogen of an antigenic PCSK9 peptide and optionally an immunogenic carrier. The antigenic PCSK9 peptide can be a portion of PCSK9 comprising between 4 to 20 amino acids and, when administered to a subject, is able to lower the LDL-cholesterol level in blood of said subject. Preferably, said subject is a mammal, preferably a human. Preferably, said antigenic PCSK9 peptide is able to lower the LDL-cholesterol level by at least 2%, 5%, 10%, 20%, 30% or 50%. The antigenic PCSK9 peptide can be a portion of PCSK9 which participates in the interaction of PCSK9 with the LDL receptor. Preferably, the antigenic PCSK9 peptide is a peptide of sequence SEQ ID NO: 56, 184, 187, 332, 445, 482, 525, or 563 (SEQ ID NOs: 15, 16, 17, 18, 19, 20, 21, and 22 herein), or functional variants thereof.

The lipid metabolism-altering compound is preferably one of the following types of compounds: a non-absorbable sugar, a compound that is able to convert NH3 to NH4+ in the body, or a hydrogen-generating compound. It should be understood that the lipid metabolism-altering compound can have one of these properties, several, or all of these properties, depending upon the specific compound. Preferably, the lipid metabolism-altering compound also has the ability to reduce and/or eliminate inflammation. It should also be understood that the lipid metabolism-altering compound is not a steroid or statin. The lipid metabolism-altering compound can further be used by itself (i.e. without the cholesterol-lowering agent) to reduce high cholesterol levels.

The lipid metabolism-altering compound is preferably lactulose, or homologues thereof. Lactulose has all of the properties described above (i.e. it is a non-absorbable sugar, a compound that is able to convert NH3 to NH4+ in the body, a hydrogen-generating compound, and reduces/eliminates inflammation). Lactulose is a synthetic sugar that is a disaccharide of a molecule of fructose and a molecule of galactose. It is currently indicated for constipation treatment or for hepatic encephalopathy in removing ammonia from blood. Lactulose is non-absorbable and fermented by intestinal bacteria, resulting in the production of hydrogen. A lactulose hydrogen breath test has previously been used to detect irritable bowel syndrome by detecting an abnormal amount of hydrogen in the breath. However, the creation of hydrogen in the present invention is a positive effect of the compound so that inflammation can be treated. Lactulose can be administered in doses from 40 mL to over 1000 mL per day; however, due to the synergism with the cholesterol-lowering agent, a lower dose can be preferred.

When the lipid metabolism-altering compound is a non-absorbable sugar, it can also be any other monosaccharide, polysaccharide, or other non-saccharide sweetener, such as, but not limited to, glucose, galactose, fructose, mannitol, inulin, sucralose, aspartame, dextrose, maltodextrin, homologues, or combinations thereof. Inulin in particular is composed of a heterogeneous collection of fructose polymers, is not absorbed in the blood, and elevates the amount of H2 in mice with oral administration. The non-absorbable sugar can be administered in amounts similar to lactulose.

When the lipid metabolism-altering compound is a compound that is able to convert NH3 to NH4+ in the body, i.e. upregulate levels of ammonium, it can be any of the sugars listed above, or any other suitable compound that is able to convert NH3 to NH4+. Ammonium (NH4+) is generally produced when ammonia (NH3) reacts with proton donors (compounds that can donate a hydrogen ion). Therefore, any compound that can donate a proton can be used to convert NH3 to NH4+. Most preferably, lactulose is used to convert NH3 to NH4+.

The lipid metabolism-altering compound can be a hydrogen-generating compound that acts to generate an increase of hydrogen in the body by several different methods, such as, but not limited to, releasing hydrogen in the body, or inducing production of hydrogen in the body. The hydrogen-generating compound can alternatively not alter lipid metabolism but rather reduce or eliminate inflammation, and especially inflammation experienced when administering a cholesterol-lowering agent. The generation of hydrogen can occur anywhere in the body as desired, and can be tailored to occur in a specific site. The hydrogen-generating compound can be H2 molecules, or a composition that includes a releasable H2 moiety. Alternatively, the hydrogen-generating compound can be a compound that induces hydrogen to be released by the body itself or by another compound in the body. For example, the hydrogen-generating compound can be any compound that induces bacteria in the stomach to release or produce H2. The hydrogen-generating compound can also be a H2 infused liquid that can be a drink or administered by intravenous infusion, or any other method described herein. Combinations of any of the methods of generating hydrogen in the body can also be used.

The most preferred combination of the composition of the present invention is statins and lactulose, and more specifically, atorvastatin and lactulose.

It should be understood that the mechanism of action of the lipid metabolism-altering compound can be through the conversion of NH3 to NH4+ in combination with or in the alternative to the generation of hydrogen. The same compounds can accomplish both mechanisms, or different compounds can be used for the lipid metabolism-altering compound to accomplish each individual mechanism when and where desired. The mechanism of action is further described below.

The lipid metabolism-altering compound can be delivered at the same time as the cholesterol-lowering agent, or at different times. The lipid metabolism-altering compound can be contained within its own dosage form, within the dosage form together with the cholesterol-lowering agent (i.e. a capsule containing the lipid metabolism-altering compound and the cholesterol-lowering agent), or within the dosage form itself (i.e. a capsule coating that includes the lipid metabolism-altering compound, with the cholesterol-lowering agent within the capsule). The composition of the present invention can be tailored to provide different release profiles as needed or desired for a particular patient, such as, but not limited to, sustained release, prolonged release, or immediate release. The lipid metabolism-altering compound and the cholesterol-lowering agent can each have the same release profiles or different release profiles.

The cholesterol-lowering agent and lipid metabolism-altering compound act in a synergistic manner. Therefore, the cholesterol-lowering agent is preferably present in an amount that is lower than the normal effective dose. The lipid metabolism-altering compound can also be present in an amount that is lower than the normal effective dose. In other words, by combining the cholesterol-lowering agent with the lipid metabolism-altering compound, the effective amount needed can be reduced, which in turn reduces unwanted side effects. Therefore, the cholesterol-lowering agent and the lipid metabolism-altering compound can be present in synergistically effective amounts. This combination also allows for the use of cholesterol-lowering agents that have previously been thought to be too toxic. Hydrogen generated by the hydrogen-generating compound lactulose has showed an additive effect on pregabalin-mediated suppression of pain associated factors (Substance P and nerve growth factor (NGF)) in mice.

The mechanism of action of the present invention can be as follows, especially with respect to inflammation, but also with respect to lowering cholesterol. The hydrogen that is generated from the hydrogen-generating compound affects the total positive/negative charge of neuron cell surfaces to increase their sensitivity to the cholesterol-lowering agent. Other mechanisms of action can alternatively or additionally be present. For example, the hydrogen-generating compound in combination with the cholesterol-lowering agent can also reduce excessive levels of NGF and Substance P that are present due to inflammation. The combination also reduces or down-regulates expression of IL-1β, TNF-α, and PGE2, all mediators of inflammation and pain.

Additionally and/or alternatively, especially with respect to lowering cholesterol, when the lipid metabolism-altering compound is a compound that is able convert NH3 to NH4+, it is able to convert toxic ammonia (NH3) derived from gut bacteria to ammonium ion (NH4+), both in the gut as well as systemically throughout the body. In other words, the combination of the cholesterol-lowering agent with the lipid metabolism-altering compound has an effect at least in the gut, and preferably the whole body, by either of the above distinct mechanisms alone or in combination. In addition, these mechanisms are not mutually exclusive so that cholesterol can be lowered while reducing inflammation. This is especially beneficial when the cholesterol-lowering agent is a statin that would otherwise cause inflammation.

The composition of the present invention can be used for treating many different diseases and conditions in which high cholesterol and/or inflammation is associated. These diseases and conditions can be, but are not limited to, acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, interstitial cystitis, atherosclerosis, allergies, myopathies, leukocyte defects, cancer, endometriosis, and multiple sclerosis. Any of these diseases can be treated according to the methods detailed below.

The present invention provides for a method of lowering cholesterol, by administering a synergistically effective amount of the composition including the cholesterol-lowering agent and the lipid metabolism-altering compound of the non-absorbable sugar, compound that converts NH3 to NH4+, or hydrogen-generating compound to an individual. The cholesterol-lowering agent can be any of those described above, and the lipid metabolism-altering compound can be any of those described above. Administration can be oral, by injection, topical, or any other administration profile described herein.

The cholesterol-lowering agent and the lipid metabolism-altering compound preferably act synergistically to lower cholesterol and reduce inflammation. Specifically, hydrogen generated by the lipid metabolism-altering compound (especially when a hydrogen-generating compound) suppresses inflammation at the artery wall and also decreases reactive oxygen species (ROS) produced by inflammation. Ox-LDL cholesterol is lowered and atherosclerotic plaque is reduced. Examples 1 and 4 show that administering atorvastatin and lactulose to mice lowers Ox-LDL cholesterol and prevents atherosclerosis. Example 3 shows that lactulose can prevent fatty liver development and suppress blood Ox-LDL caused by a high fat diet. By performing this method, side effects normally experienced by patients who are administered cholesterol-lowering agents can be reduced by reducing the dose needed due to be effective.

The present invention also provides for a method of lowering cholesterol and reducing inflammation associated with cholesterol-lowering agents, by administering synergistically effective amounts of the lipid metabolism-altering compound and the cholesterol-lowering agent to an individual, reducing LDL levels, and preventing inflammation. Not only can the lipid metabolism-altering compound reduce high cholesterol levels (as further described below), the lipid metabolism-altering compound counteracts, reduces, treats, and prevents the unwanted side effect of inflammation caused by many cholesterol-lowering agents, especially statins. Because the compounds can be administered synergistically, lower amounts can also be administered, further reducing the side effect of inflammation in the first place. Therefore, by administering a composition to reduce cholesterol, an anti-inflammation effect is generated, and likewise, by administering a composition to reduce inflammation, a cholesterol lowering effect is generated.

The present invention also provides for a method of preventing atherosclerosis by administering an effective amount of the composition including the cholesterol-lowering agent and the lipid metabolism-altering compound to an individual, and reducing ox-LDL cholesterol. This method can further include reducing atherosclerotic plaque in the aorta of an individual.

The present invention further provides for a method of treating rheumatoid arthritis by administering an effective amount of the composition including the cholesterol-lowering agent and lipid metabolism-altering compound to an individual, and reducing inflammation. Rheumatoid arthritis causes the immune system to attack healthy joints, resulting in inflammation, pain, swelling, and destruction of cartilage and bone. The cholesterol-lowering agent and the lipid metabolism-altering compound work synergistically to reduce the amount of inflammation.

The present invention provides for a method of lowering cholesterol, by administering a synergistically effective amount of the lipid metabolism-altering compound and a lipid-lowering antibody, and reducing LDL levels. The lipid metabolism-altering compound can be any described above, and preferably lactulose. Preferably, the lipid-lowering antibody is an anti-PCSK9 MAb, and further includes the step of blocking PCSK9. The anti-PCSK9 MAb can be any compound described above or any MAb that is able to effectively block the action of PCSK9. The method can further include administering another cholesterol-lowering agent such as a statin as described above. Each of the compounds can be administered at the same time with the same dosage forms or at separate times and separate dosage times as described above.

While the combination of the lipid metabolism-altering compound and anti-PCSK9 MAb can be used to lower high cholesterol, the lipid metabolism-altering compound can also be used by itself to convert NH3 to NH4+ to protect PCSK9-mediated LDL-receptor (LDLR) degradation and lower cholesterol and LDL levels. This can be advantageous when an injection is not desired by the user, as the hydrogen-generating compound can be administered orally. Therefore, the present invention also provides for a method of lowering cholesterol by administering an effective amount of a lipid metabolism-altering compound, converting NH3 to NH4+, and reducing LDL levels.

The lipid metabolism-altering compound can also be used on its own in a method of reducing oral odor by administering the lipid metabolism-altering compound to an individual, and reducing hydrogen sulfide producing bacteria. The bacteria that are reduced can be found in the mouth or in the intestines. Essentially, volatile sulfur compounds are reduced by the administration of the lipid metabolism-altering compound. Example 2 shows that the lipid metabolism-altering compound can reduce hydrogen sulfide present in a culture, therefore it can be used to reduce hydrogen sulfide in the body and reduce oral or other body odor.

The present invention further provides for a method of suppressing weight gain, by administering a synergistically effective amount of the composition including the cholesterol-lowering agent and the lipid metabolism-altering compound to an individual, and suppressing weight gain. This method is further described in EXAMPLE 4, which shows that lactulose can prevent weight gain on a fatty diet, and that lactulose in combination with a statin can significantly suppress weight gain on a fatty diet. Lactulose and statins are also shown to suppress hyperlipidemia and have an adiposity protective effect. Lactulose and statins are shown to prevent metabolic syndrome effects.

The cholesterol-lowering agent can be any of those described above, and the lipid metabolism-altering compound can be any of those described above. Most preferably, the cholesterol-lowering agent is atorvastatin and the lipid metabolism-altering compound is lactulose. Lactulose is particularly advantageous because while it is a sugar, it does not pass through the intestine and not absorbed into the blood stream, and therefore provides zero calories and does not contribute to weight gain. All of the lactulose in the gut are digested by bacteria and the end products, including hydrogen and short chain fatty acids, are responsible for benefits of ingestion of lactulose. Administration can be oral, by injection, topical, or any other administration profile described herein.

Lactulose is also advantageous in that it can provide a much better probiotic action than Lactobacillus (yogurt bacteria) and changes the bacterial flora in the gut from bad to good. Probiotics are microorganisms that are introduced into the body due to their beneficial qualities. Examples of probiotics are bacteria and yeast in supplements and food. Probiotics are meant to maintain the health of the digestive system and lining of the intestines and aid the immune system. The natural balance of bacteria can become disturbed after an infection or the use of antibiotics, and it can be helpful to take probiotics to repopulate the digestive system with beneficial bacteria. The lactulose of the present invention is a true probiotic, i.e. a substance that has a beneficial effect on the intestinal microbial balance of the individual taking the composition. The lipid metabolism-altering compound, preferably lactulose, can induce the growth of beneficial bacterial flora in the gut and that induces the flora to generate systemic hydrogen, which, in turn, positively affects the redox value systemically of the person and reduces inflammation, so it is a true probiotic unlike most substances that claim to be probiotic that really just provide dead culture to the body. The lipid metabolism-altering compound actually, in essence, feeds and induces the flora in the gut to grow, which, in turn, induces generation of hydrogen by the flora and the bacteria in the gut, which positively affects the redox value of the body (it raises the levels of anti-oxidant hydrogen in the body). Reducing the number of free radicals (and thus the redox value) to reduce oxidative stress is the goal of taking antioxidants (Lobo, et al., Pharmacogn Rev. 2010 July-December; 4(8): 118-126). This can be achieved through administering the lipid metabolism-altering compound in a beverage form, in a drink form, as an additive to drinks, as an additive to food, or in any of the other forms as described above. By administering the lipid metabolism-altering compound of the present invention over time, there is a cumulative probiotic effect.

The cholesterol-lowering agent and the lipid metabolism-altering compound preferably act synergistically to lower cholesterol, reduce inflammation, and suppress weight gain in individuals who eat a high fat diet. Specifically, hydrogen generated by the lipid metabolism-altering compound suppresses inflammation at the artery wall and also decreases reactive oxygen species (ROS) produced by inflammation. Ox-LDL cholesterol is lowered and atherosclerotic plaque is reduced. By performing this method, side effects normally experienced by individuals who are administered cholesterol-lowering agents can be reduced by reducing the dose needed due to be effective. The lipid metabolism-altering compound can also produce these results without the cholesterol-lowering agent.

The compounds of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

The purpose of this project is to investigate the combination effects of atorvastatin (LIPITOR®, Pfizer) and hydrogen (as provided by lactulose) to arterial sclerosis induced in a mice model.

Background

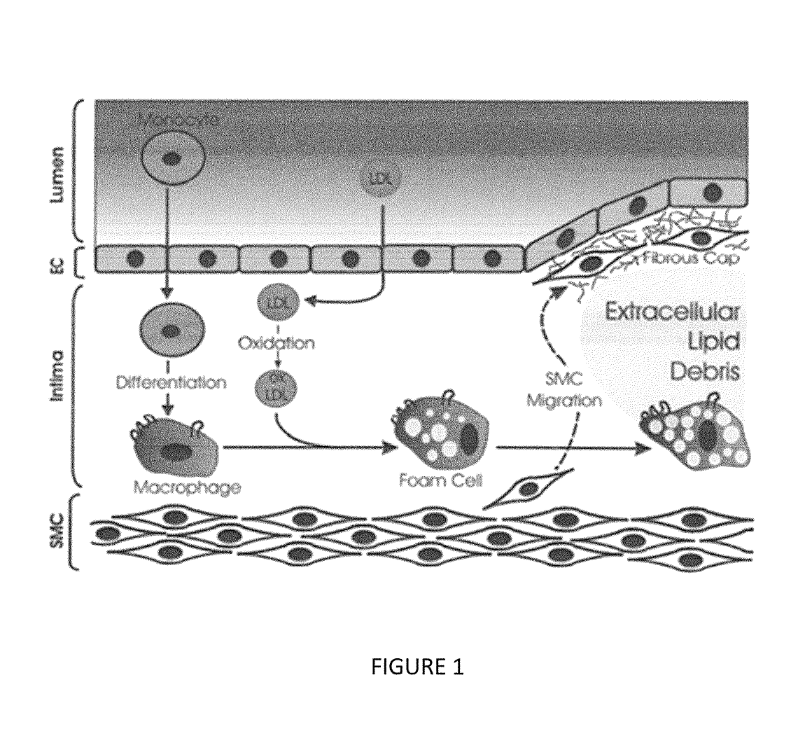

Bacterial flora in the intestine produces hydrogen by digesting Lactulose, continuously. Hydrogen has anti-oxidant effects. When Low-density-lipoprotein receptor (LDLR) knockout mice are fed with high fat diet, this strain of mice show elevated level of Oxidative LDL (ox-LDL) which is a major factor for athelosclerosis (see FIG. 1). Statins are HMG-CoA reductase inhibitors which decrease atherosclerosis by lowering LDL cholesterol in human except high concentration atorvastatin. Atorvastatin (Lipitor, Pfyzer) has been the best-selling drug among 7 kinds of statins in the United States.

Hypothesis

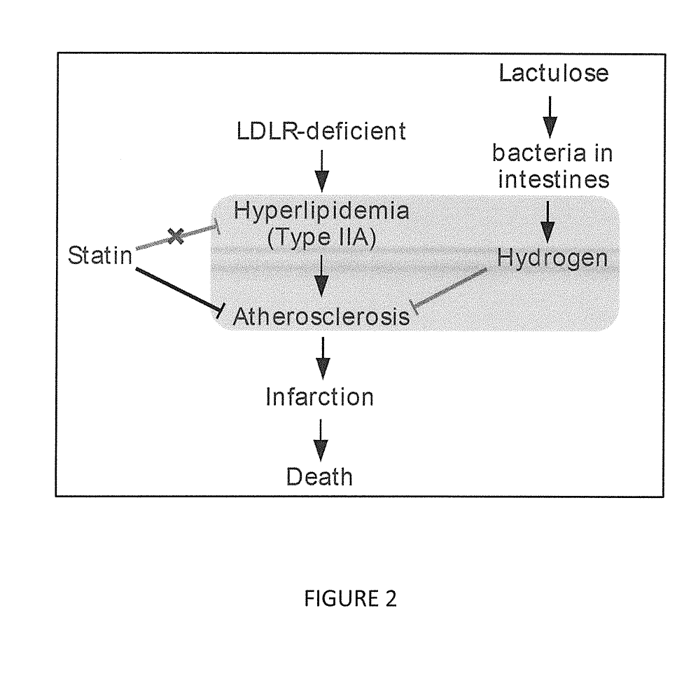

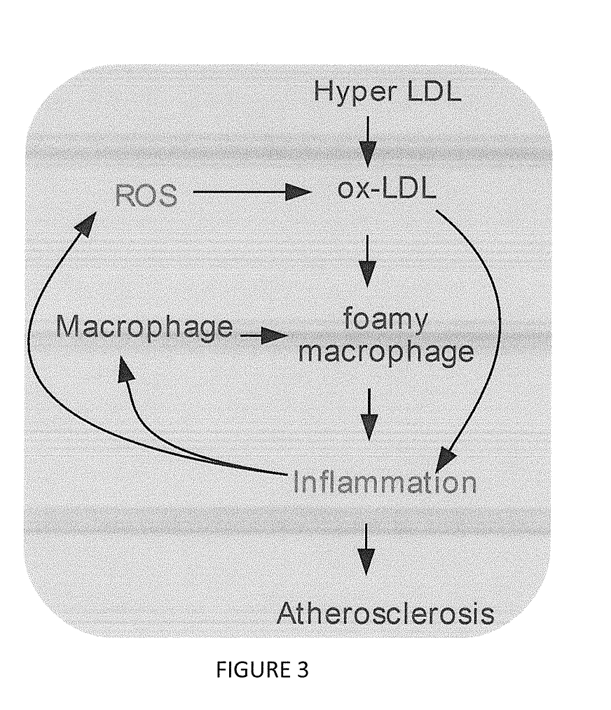

It was hypothesized that the combination of Atorvastatin and Hydrogen prevents the atherosclerosis in mice hyperlipidemia/atherosclerosis model (FIG. 2) by two pathways (FIG. 3).

(1) Hydrogen suppresses inflammation at artery wall.

(2) Decrease reactive oxygen species (ROS) produced by inflammation.

Methods

Two-months-old LDLR knockout mice on a C57BL/6J background are used in this project. They are fed with a chow diet (CHOW) as a control diet or western diet (WEST) as a high fat diet with or without atorvastatin (ATV). To check the hydrogen or lactulose effects, CHOW and WEST groups are separated into three groups, Control group(Cont.), Hydrogen group(Hydr): hydrogen was added to their drinking water, Lactulose group (Lact): Lactulose was ingested twice in a day.

All mice are sacrificed and blood was collected and serum obtained. Aorta was extracted and atherosclerosis was evaluated Histological analysis.

Results

Experiment 1

At the age of 12 week, all mice were sacrificed. No plaque at the artery wall was observed at age of 12 week old, indicating that longer exposure with fat enriched diet is required.

### Example 2

### Purpose

Investigate the effects of Hydrogen water rinse on oral odor (bad breath).

Background

The main factors for oral odor are derived from Volatile Sulfur Compounds (VSC) which are produced by oral bacteria. Hydrogen sulfide (H2S) is one of the major VSC and toxic, whereas dimethyl sulfide (DMS), another type of VSC, produced from oral bacteria is not harmful to the host.

Bacteria in intestines also produce hydrogen sulfide. Based on the reports from Japanese consumers who used hydrogen-water, odor of intestinal gas (fart) diminished after drinking hydrogen-water on regular basis.

Hypothesis

Oral odor is reduced by mouth wash with hydrogen water.

Methods

Incubate one of the periodontal bacteria for 4 hours with/without hydrogen and measure the hydrogen sulfide concentration.

Culture F. nucleatum (oral bacteria that produce H2S) overnight.

Incubate F. nucleatum in H2 or N2 condition for 4 hours.

Keep supernatant (3 ml) in 15 ml conical tube at 4° C.

Measure the H2S concentration in the conical tube (N=5, 1:10 dilution).

Results

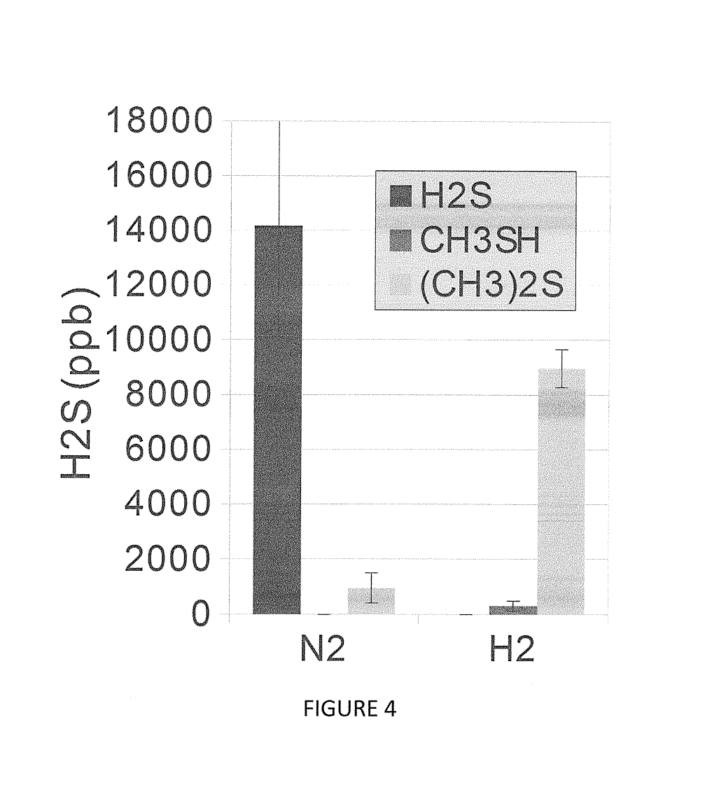

Hydrogen sulfide tends to be reduced by adding hydrogen to the culture condition. While the F. nucleatum cultured with H2 showed the elevated production of dimethyl sulfide (DMS) instead of H2S, such DMS produced by bacteria should not cause any harmful effect to the host. See FIG. 4.

### Example 3

### Materials and Methods

Animals

Low density lipoprotein receptor (LDLR) knockout mice and their wild type (C57BL6/J), were purchased from Jackson Laboratories (Bar Harbor, Me., USA). Male LDLR knockout mice (9-w old male) received control standard diet (Picolab, MO, USA; diet #5053) or high fat diet (Tekland Custom Diet, WI, USA; diet #TD88137). The LDLR mice received high fat diet were further randomly divided into four groups; G1) 20 mg/kg day atorvastatin group, G2) 400 mg/day lactulose group, G3) combination group, and G4) negative control group (no atorvastatin or lactulose). Atorvastatin was supplemented in the diet and lactulose was administered by oral gavage, twice a day. Wild type mice were maintained on the standard diet. All mice were fed with above noted respective diet for 12 weeks and sacrificed for post mortal analyses.

Blood was collected by heart puncture at left ventricular on the sacrificed mice, followed flushing the blood circulation with PBS, and the aorta and liver lobes were removed for laboratory evaluations.

Lipoproteins

Blood was drawn from heart in the absence of anti-coagulant agents, and the serum was prepared. High-density lipoprotein cholesterol and low-density lipoprotein (LDL) cholesterol were determined by a commercial colorimetric assay (Sigma-Aldrich, MO, USA). Oxidative-LDL was measured by ELISA (Uscn Life Science, TX, USA).

Histology

Aortas and liver lobes were frozen in OCT compound (Sakura finetek, Japan).

Cryosections of aortas and liver lobes were stained with hematoxylin and eosin. Atherosclerosis legions were evaluated by Oil-Red 0 staining.

Macrophages were stained with the rat anti-mouse macrophage monoclonal antibody against CD68 (IgG2a, MCA1957GA, Serotec Inc.) followed by goat-anti-rat IgG (IgG:HRP STAR72; Serotec Inc.) and VECTASTAIN® Elite ABC-Peroxidase Kit (Vector Laboratories). The positive staining for CD68 was detected by Peroxidase-specific DAD color development (Vector Laboratories).

Results

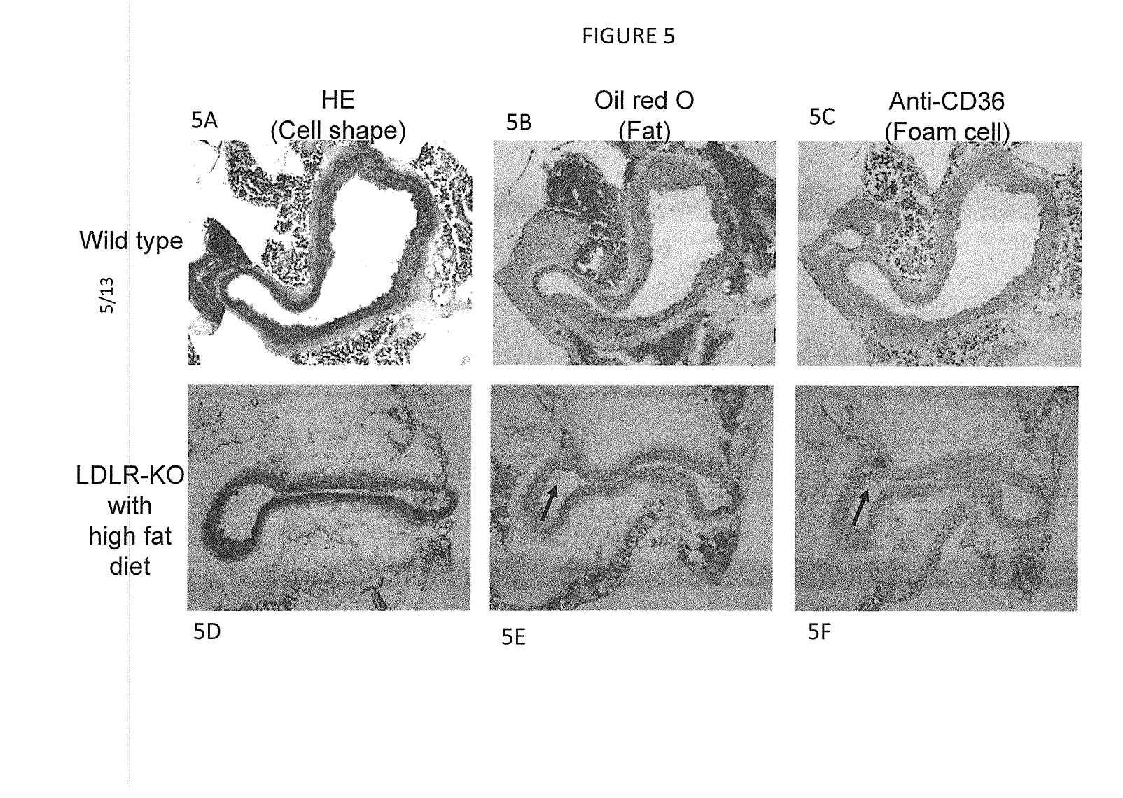

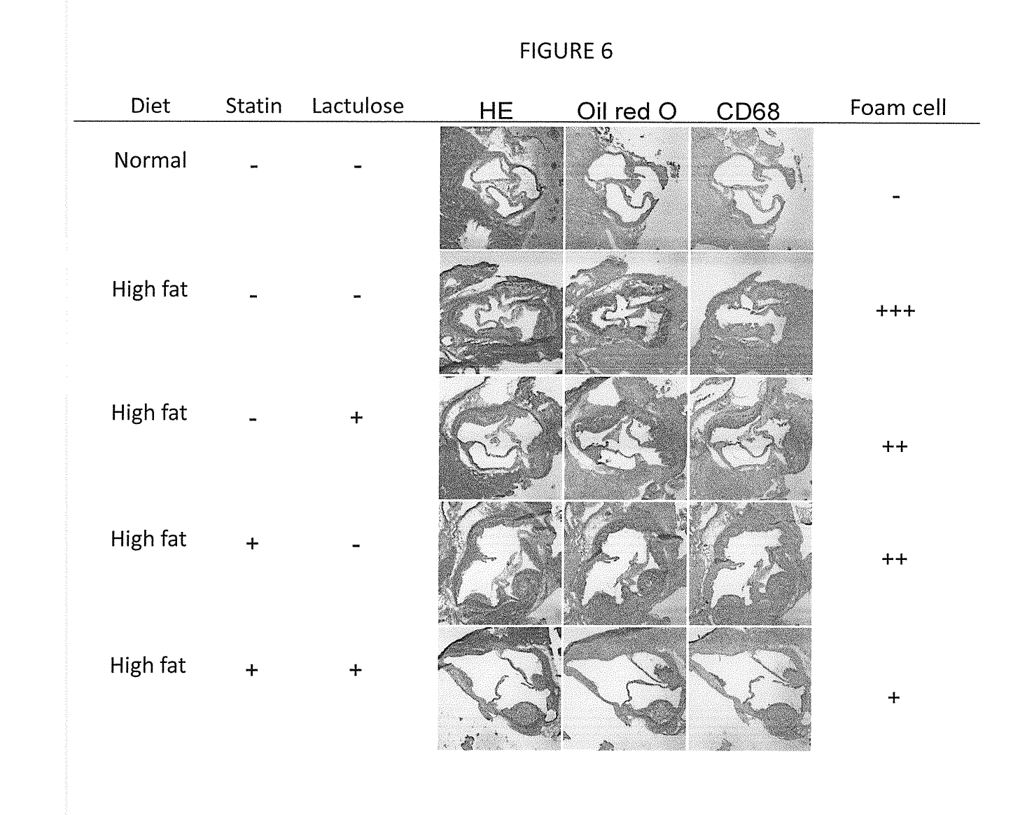

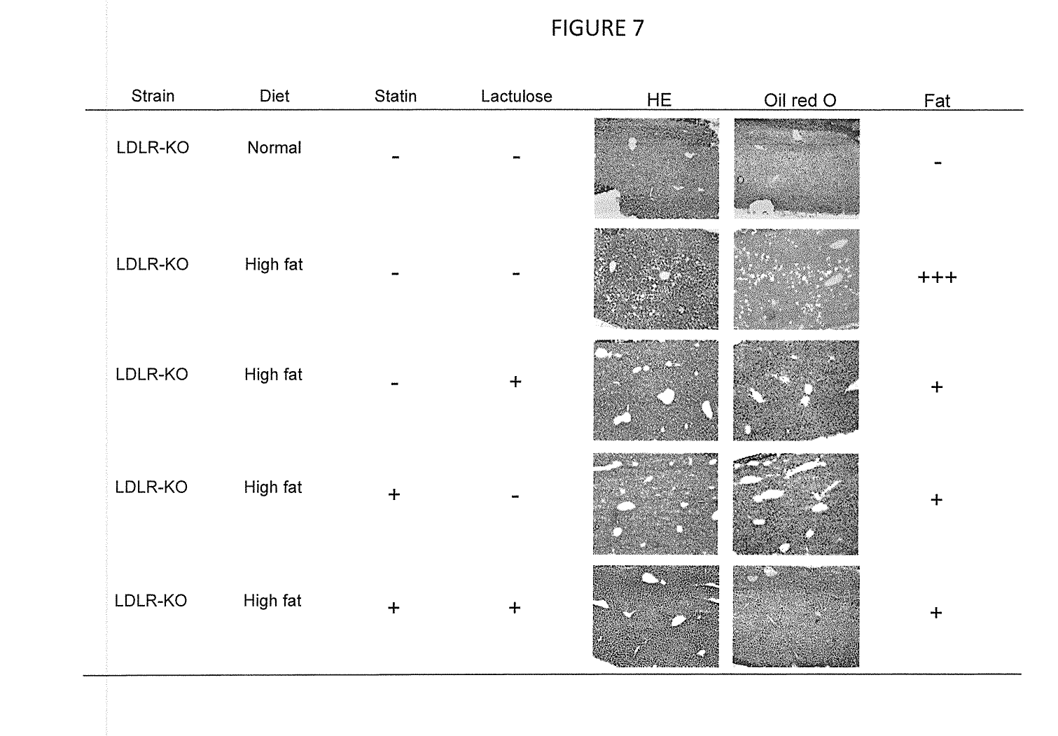

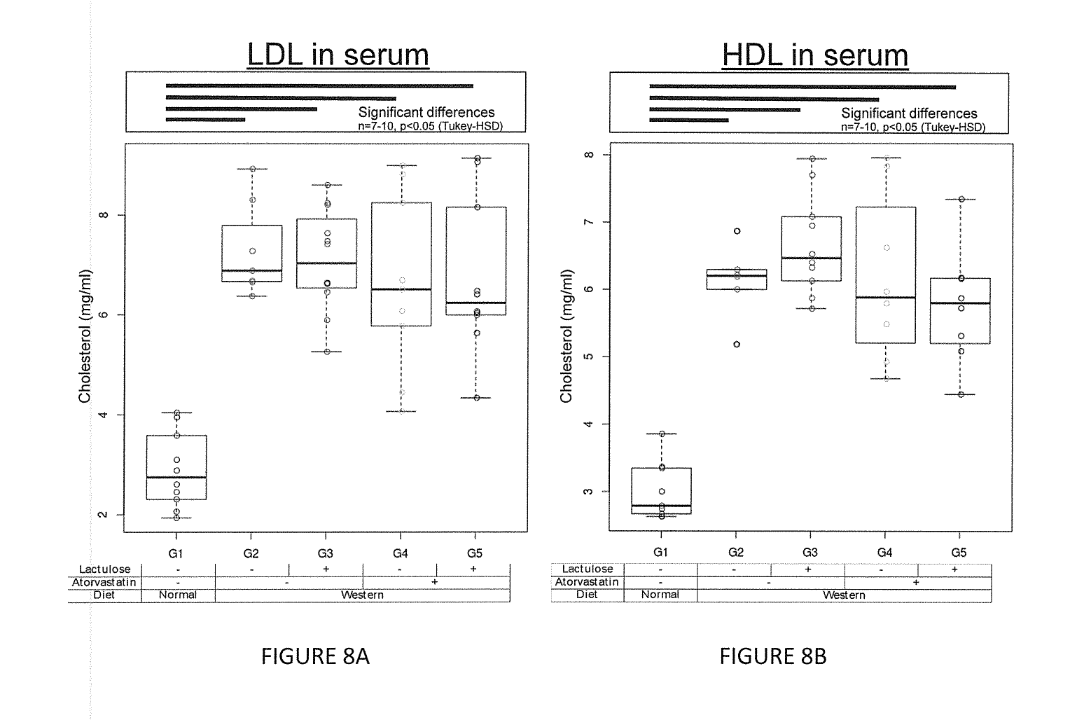

FIGS. 5A-5F show that the atherosclerosis region was detected in aorta of LDLR-KO mice by feeding them with a high fat diet for 3 months. FIG. 6 is a chart showing that the level of plaque deposition was very modest in LDLR-KO mice that received the high fat diet. Longer feeding (>6 months) will be required to detect the combination effects of lactulose and statin. FIG. 7 is a chart showing that lactulose has preventative effects on fatty liver development. FIG. 8A shows that the amount of HDL (good cholesterol) didn't change by administration of either the statin or lactulose. FIG. 8B shows that LDLR-KO mice show higher LDL (bad cholesterol) levels when they were fed with a high fat diet. Administration of the statin suppressed LDL in fat-diet treated mice. However, lactulose showed no effects on LDL by itself or no additive effects on LDL as combined with the statin.

In conclusion, atherosclerosis legion was detected in the aorta. However, the period of fat-diet feeding was not enough to detect the lactulose and statin combination effects. There were remarkable preventative effects for development of fatty liver lesion by lactulose, showing that lactulose has beneficial effects on fat metabolism in the liver. It was previously found that lactulose by itself can suppress blood Ox-LDL that was caused by a fat diet. However, the amount of blood LDL did not change by lactulose.

### Example 4

### Materials and Methods

Animals: Homozygous LDL receptor knockout (LDLr−/−; C57BL/6) mice were purchased from the Jackson Laboratory (Bar Harbor, Me.). This genetically manipulated mouse strain is most commonly used for the pre-clinical study of high-fat diet induced hyperlipidemia and atherosclerosis. Nine weeks of age mice were separated into five groups (Groups #1-#5) and fed with normal diet (Group #1; Picolab, 5053) or high fat diet (Group #2-#5; HFD; Harlan Teklad, TD 88137) for 12 weeks. Atorvastatin (Lipitor) was added to the HFD (0.16% w/w) to Group #4 and Group #5. Lactulose (3 g/kg/day, in water solution) was orally administered every 12 hours to Group #3 and Group #5. The LDLR-KO mice were fed for three months with a normal or western diet, with or without lactulose, and with or without atorvastatin.

Analysis of serum samples: Lipids, including LDL and HDL, as well as oxidized low density lipoprotein (Ox-LDL) present in serum was measured using an ELISA kit (Cell biolabs).

Analysis of atherosclerotic lesion: Development of atherosclerotic plaque in the lumen of aorta (from aortic root to the iliac bifurcation) was determined by oil red O staining. After removal of fat that is deposited out side of aorta, the aorta was dissected under the dissection microscope, and cut to open the lumen longitudinally from the aortic root to the iliac artery. The resulting section was immersed in the fixative for 12 hours and rinsed with PBS which was, then, stained with Oil red O (Sigma). After washing the Oil red O-reacted aortas with 60% isopropanol, the digital image of Oil-red-stained lumen was captured using a camera attached to dissection microscope (Nikon, SMZ745T). The area stained with Oil red O was quantified using ImageJ software (NIH).

Results

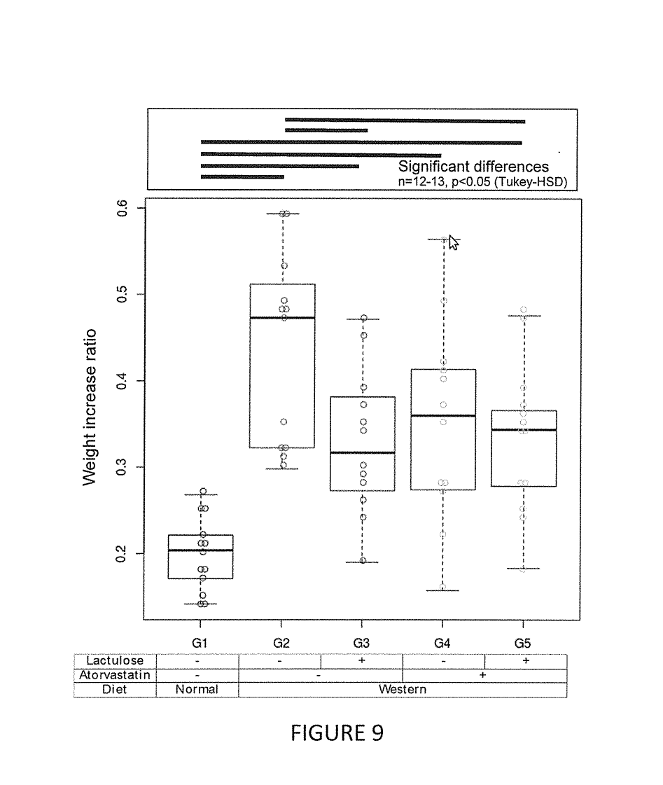

Results of the experiment are shown in FIG. 9. Mice weight significantly increased with a western diet, even when they were treated with a statin (G2 and G4 (statin) compared to G1; n=7-9, *p<0.05; Tukey-HSD). Groups that were administered lactulose showed a moderate weight increase compared to groups that were not given lactulose (G3 and G5 compared to G2 and G4, respectively). The combination of lactulose and statin significantly suppressed weight increase (G2 compared to G5).

It is reported that statins (atorvastatin) do not work in mice to reduce their hyperlipidemia. The present invention shows that the combination of lactulose and atorvastatin suppresses hyperlipidemia. Based on these unexpected results, lactulose is shown to have an adiposity protective effect. In other words, lactulose can unexpectedly suppress weight gain (obesity) induced by a high fat diet in LDLR-KO mice and also unexpectedly works synergistically with atorvastatin to suppress weight gain while reducing unwanted side effects of atorvastatin. A completely novel mechanism mediated by lactulose elicits the suppression of hyperlipidemia in concert with atorvastatin. A similar effect is produced by lactulose on xPCSK9 MAb.

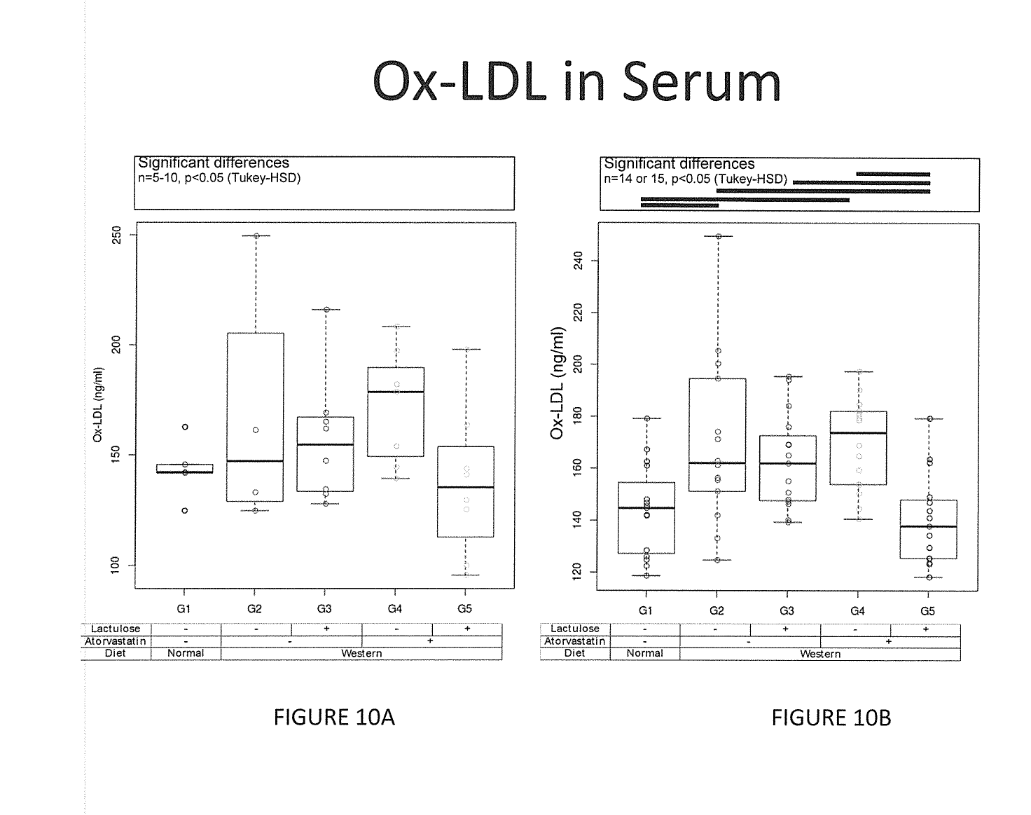

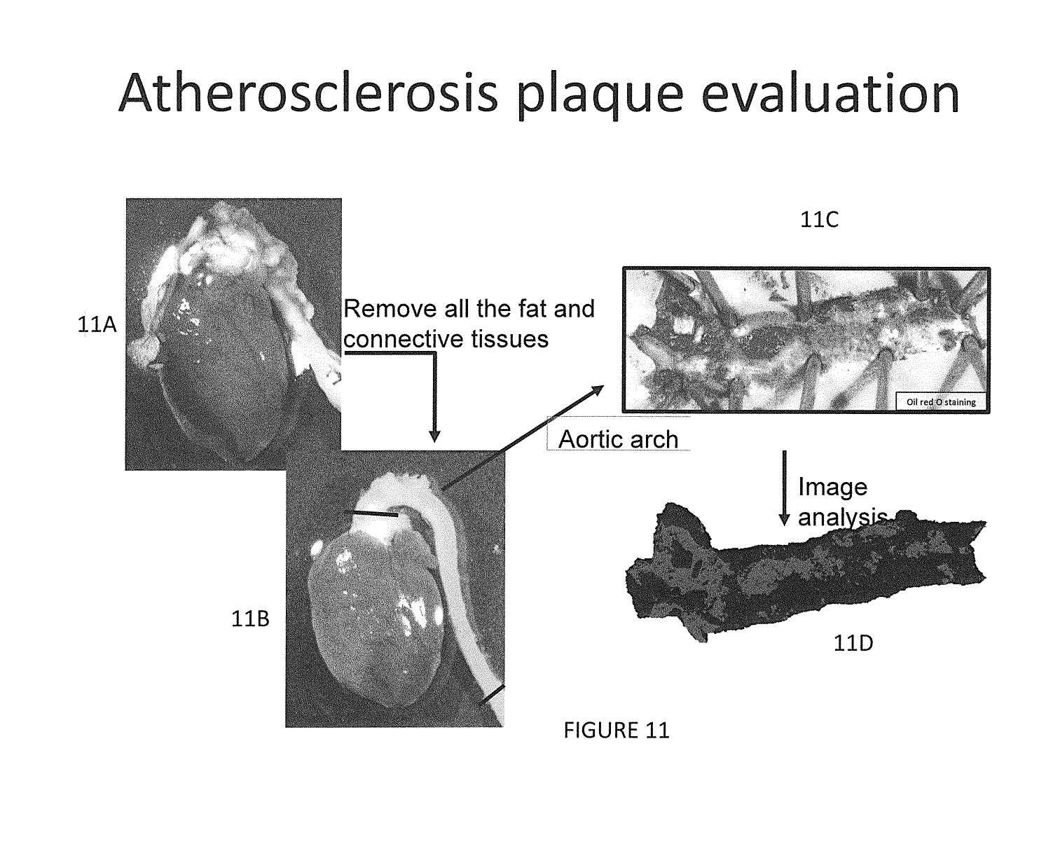

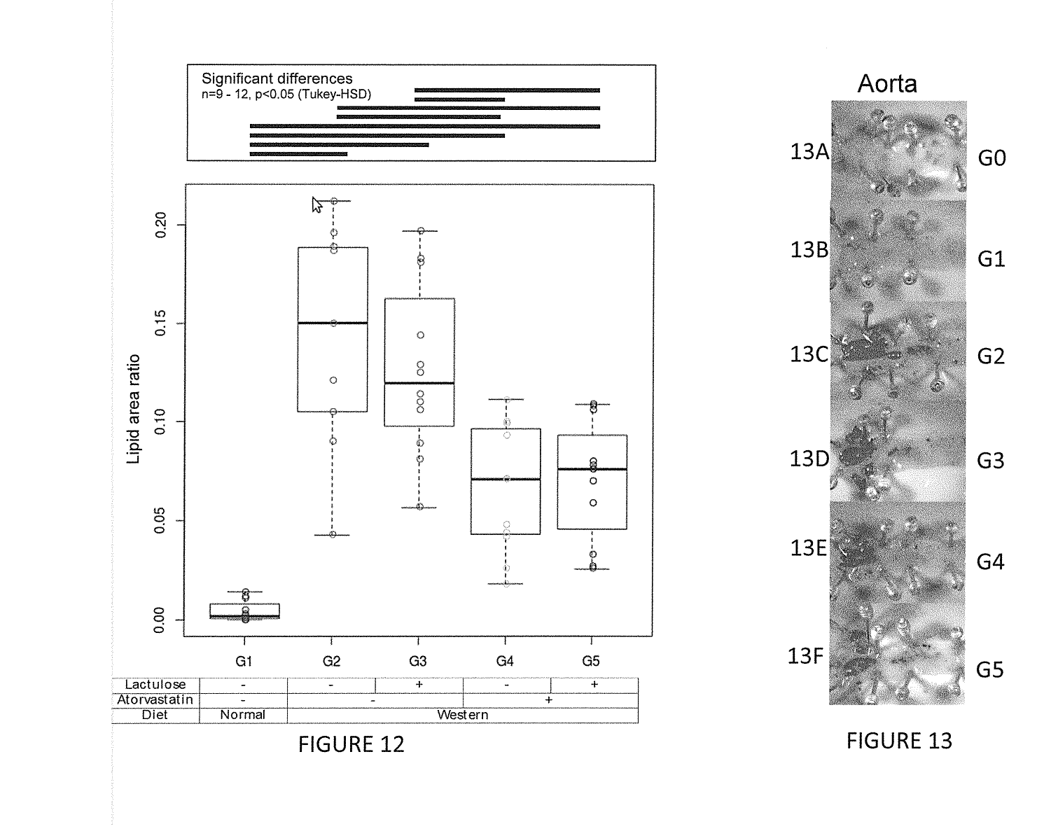

Lactulose can also suppress the oxidized LDL concentration and this can reduce atherosclerotic plaque. FIGS. 10A and 10B show the amount of Ox-LDL in mice at 1.5 months and 3 months with different diets, both with and without the combination of a statin and lactulose. Lactulose was able to suppress Ox-LDL alone and in combination with the statin. FIGS. 11A-11D show photographs of the evaluation of atherosclerosis plaque in the mice. As shown in FIG. 12 and in FIGS. 13A-13F, a western diet increased the atherosclerotic plaque at the LDLR-KO mice aorta (G2-G5).

Lactulose is considered a prebiotic. It modulates the beneficial bacterial flora that produce short-chain fatty acid (SCFA) in the gut. Recently, it was reported that the gut microbiota suppresses insulin-mediated fat accumulation via the SCFA receptor GPR43 (Nat Commun. 2013). Together with the results herein, it is shown that preventive effects can be achieved for metabolic syndrome (obesity, atherosclerosis, diabetes, fatty liver, etc.) by SCFA metabolized from lactulose.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for treating high cholesterol, comprising synergistically effective amounts of a cholesterol-lowering agent and a lipid metabolism-altering compound chosen from the group consisting of a non-absorbable sugar, a compound that converts NH3 to NH4+, hydrogen-generating compound, and combinations thereof.

2. The composition of claim 1, wherein said cholesterol-lowering agent is a statin chosen from the group consisting of atorvastatin, cerivastatin, fluvastain, lovastatin, mevastatin, pitavastatin, pravastatin, rosuvastatin, and simvastatin.

3. The composition of claim 2, wherein said statin is administered in an amount of 10 to 80 mg/day.

4. The composition of claim 1, wherein said cholesterol-lowering agent is a lipid-lowering antibody.

5. The composition of claim 4, wherein said lipid-lowering antibody is anti-PCSK9 MAb.

6. The composition of claim 1, wherein said lipid metabolism-altering compound is lactulose or homologues thereof and is in an amount of 40 mL to 1000 mL per day.

7. The composition of claim 1, wherein said non-absorbable sugar is chosen from the group consisting of lactulose, glucose, galactose, fructose, mannitol, inulin, sucralose, aspartame, dextrose, maltodextrin, homologues, and combinations thereof.

8. The composition of claim 1, wherein said hydrogen-generating compound is chosen from the group consisting of H₂ molecules, a prodrug releasing H₂, a compound that causes the release of H₂ within the body, an H₂ infused liquid, and combinations thereof.

9. The composition of claim 1, wherein said compound that converts NH3 to NH4+ is lactulose or homologues thereof.

10. The composition of claim 1, wherein said composition is in a dosage form is chosen from the group consisting of said lipid metabolism-altering compound in its own dosage form, said lipid metabolism-altering compound and said cholesterol-lowering agent in the same dosage form, or said lipid metabolism-altering compound within a coating of the dosage form and said cholesterol-lowering agent within the dosage form.

11. A composition for treating high cholesterol, comprising synergistically effective amounts of a statin chosen from the group consisting of atorvastatin, cerivastatin, fluvastain, lovastatin, mevastatin, pitavastatin, pravastatin, rosuvastatin, and simvastatin, and lactulose.

12. The composition of claim 11, further defined as synergistically effective amounts of atorvastatin and lactulose.

13. A method of lowering cholesterol, including the steps of:
administering a synergistically effective amount of a composition including a cholesterol-lowering agent and a lipid metabolism-altering compound chosen from the group consisting of a non-absorbable sugar, a compound that converts NH3 to NH4+, a hydrogen-generating compound, and combinations thereof to an individual; and
lowering cholesterol in the individual.

14. The method of claim 13, further including the steps of suppressing inflammation at the artery walls of the individual and decreasing reactive oxygen species produced by inflammation.

15. The method of claim 13, further including the steps of lowering Ox-LDL, HDL, and LDL levels.

16. The method of claim 13, further including the step of reducing atherosclerotic plaque in aortas of the individual.

17. The method of claim 13, further including the step of reducing side effects of cholesterol-lowering agents.

18. The method of claim 17, wherein said reducing side effects step further includes preventing, reducing, and/or treating inflammation.

19. The method of claim 13, further including the step of elevating ammonium ion levels in individual, maintaining LDLR levels, and capturing and destroying LDL in the liver.

20. The method of claim 13, further including the step of suppressing weight gain in the individual.

21. The method of claim 13, wherein the cholesterol-lowering agent is a statin chosen from the group consisting of atorvastatin, cerivastatin, fluvastain, lovastatin, mevastatin, pitavastatin, pravastatin, rosuvastatin, and simvastatin.

22. The method of claim 13, wherein the cholesterol-lowering agent is a lipid-lowering antibody.

23. The method of claim 22, wherein the cholesterol-lowering agent is further defined as an anti-PCSK9 MAb or portion thereof, and further including the step of blocking PCSK9 and reducing LDL levels.

24. The method of claim 13, wherein the lipid metabolism-altering compound is a non-absorbable sugar chosen from the group consisting of lactulose, glucose, galactose, fructose, mannitol, inulin, sucralose, aspartame, dextrose, maltodextrin, homologues, and combinations thereof.
